(19) United States
(12) Patent Application Publication
(10) Pub. No.: US 2005/0234441 A1
(43) Pub. Date: Oct. 20, 2005

(54) Guided and filtered user interface for use with an ophthalmic surgical system

(76) Inventors: Michael E. Bisch, Kirkwood, MO (US); Bruce R. Cochran, St. Charles, MO (US)

Correspondence Address: Bausch & Lomb Incorporated, One Bausch & Lomb Place, Rochester, NY, 14604-2701, US

(21) Appl. No.: 11/071,087

(22) Filed: Mar. 3, 2005

Related U.S. Application Data
(60) Provisional application No. 60/557,629, filed on Mar. 30, 2004.

(51) Int. Cl. A61B 18/18

(52) U.S. Cl. 606/38

(57) ABSTRACT

An ophthalmic surgical system 10 includes an aspiration device 12 for aspirating fluids and tissue from a surgical site 14, and a surgical handpiece 16 for performing a surgical function. A control unit 46 is connected to each of the aspiration device and the surgical handpiece for controlling the operation of each device and handpiece. A user interface 48 forming a portion of the control unit 46, guides a user by prompting the user to answer a series of questions and the control unit, controls the devices and handpieces based on the user's answers.

Priority is hereby claimed in the present nonprovisional application to Provisional Application Ser. No. 60/557,629 filed Mar. 30, 2004, in accordance with 37 CFR 1.78(a)(4).

### BACKGROUND OF THE INVENTION

1. Field of the Invention

The present invention is related to graphical user interfaces in surgical systems. More particularly, the present invention is directed to an ophthalmic surgical system having a user interface that presents a guided and filtered set-up for the surgical system.

2. Description of Related Art

Current surgical systems, particularly for ophthalmic surgery, are powerful and can be complicated to set-up properly to the desires of a surgeon. Presently, systems require the use of parameter entry screens requiring specific numbers to be entered into the screens and for the user to know what screens need to be accessed. These screens can be cumbersome and it is not always obvious to the user if all the required information for the planned surgery has been entered into the surgical system. Most systems do offer default settings, though these default settings are often much less than the optimum desired by a particular surgeon for a particular surgery.

In addition, these current systems do not direct or guide a user through system set-up, but rather rely on the user to initiate the settings that he or she desires. Furthermore, essentially all options are available to a surgeon during set-up, rather than providing what could be considered to be a preferred mode where the presentation of options is filtered. That is to say that if a surgeon initially chooses to use a peristaltic pump, the options for other pump systems are still available to the surgeon; rather than the system not presenting (i.e. filtering the options) the surgeon with options for a venturi or rotary vein pump setting.

Therefore, it would be desirable to provide a user interface for ophthalmic surgical systems which guides and filters a set-up presentation to the user to provide a simpler and easier to understand set-up procedure than has previously been known.

### BRIEF DESCRIPTION OF THE DRAWINGS

FIG. 1 is a partial block diagram of an ophthalmic surgical system in accordance with the present invention;

FIG. 2 is a flow chart of a surgical file creation presentation in accordance with the present invention;

FIGS. 3 and 4 are screens presented to the user and form a part of the user file creation presentation in accordance with the present invention;

FIG. 5 is a flow chart for customizing irrigation and aspiration settings in accordance with the present invention;

FIG. 6 is a flow chart for customizing aspiration settings in accordance with the present invention; and

FIG. 7 is a flow chart for customizing ultrasound settings in accordance with the present invention.

### DETAILED DESCRIPTION

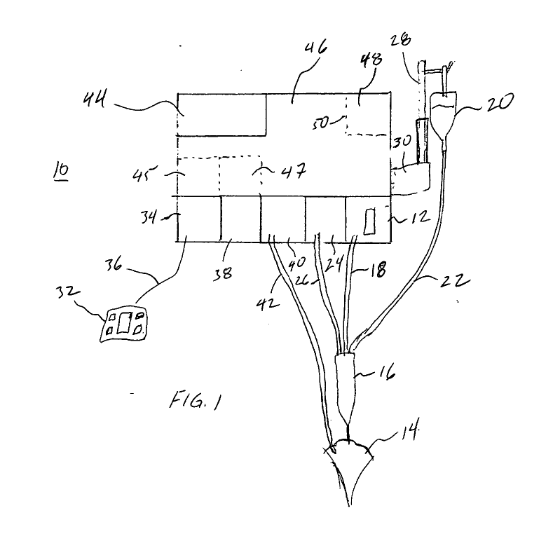

FIG. 1 shows an ophthalmic surgical system 10, which is similar to Bausch & Lomb Incorporated's Millennium™ microsurgical system. System 10 preferably includes at least one aspiration device 12, such as the venturi pump shown for aspirating fluids and tissue from a surgical site, such as eye 14. It is noted that aspiration device 12 could be other type of fluid flow devices such as a peristaltic pump or a rotary vein pump. System 10 also in one embodiment includes at least one surgical handpiece 16, such as the phacoemulsification handpiece shown for performing a surgical function, such as removing a cataract from eye 14. As shown, handpiece 16 is connected via tube 18 to pump 12 and to irrigation bottle 20 via tube 22 and is also connected to power module 24 via cable 26.

System 10 also preferably includes an irrigation pole 28 whose height can be adjusted via motor 30, as is commonly known. System 10 also typically includes a foot pedal 32 connected to a module 34 via cable 36, as shown. System 10 may also include other modules, such as module 38 for use with other instruments, such as laser probes, viteous cutters or other known ophthalmic surgical instruments, as well as a lighting module 40 connected to the eye via light-pipe 42. Hanpieces 16 include irrigation, aspiration, co-agulator, laser, vitrectomy cutter, scissors, and illumination handpieces.

Further, System 10 typically includes a display screen 44. All the surgical devices and handpieces are typically connected to and controlled by control unit 46, including a central processing unit 45 and memory 47 for controlling the operation of each device and handpiece. The present invention also includes a user interface 48 forming a portion of the control unit 46, as shown by dash-line 50, for guiding a user by prompting the user to answer a series of questions via the screen 44 and the control unit 46 controlling the devices and handpieces based on the user's answers, as described in detail below. The user interface 48 also preferably includes a filtering presentation of options to the user based on the answers provided by the user. That is to say the filtering presentation intelligently makes available only those options that are still relevant based on the previous answer provided by the user. For example if the user selects a venturi pump, the options and questions needing to be answered regarding a peristaltic pump or rotary vein pump may be filtered out of the presentation, and not be presented to the user.

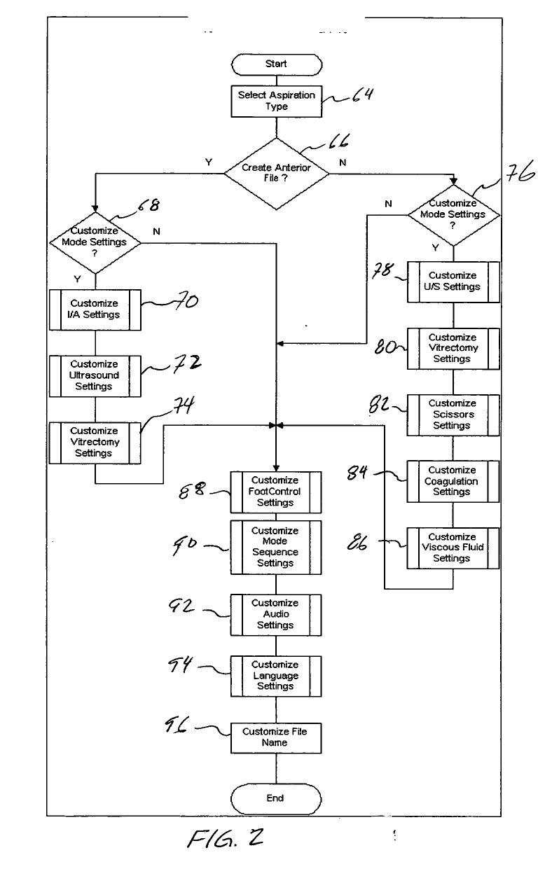

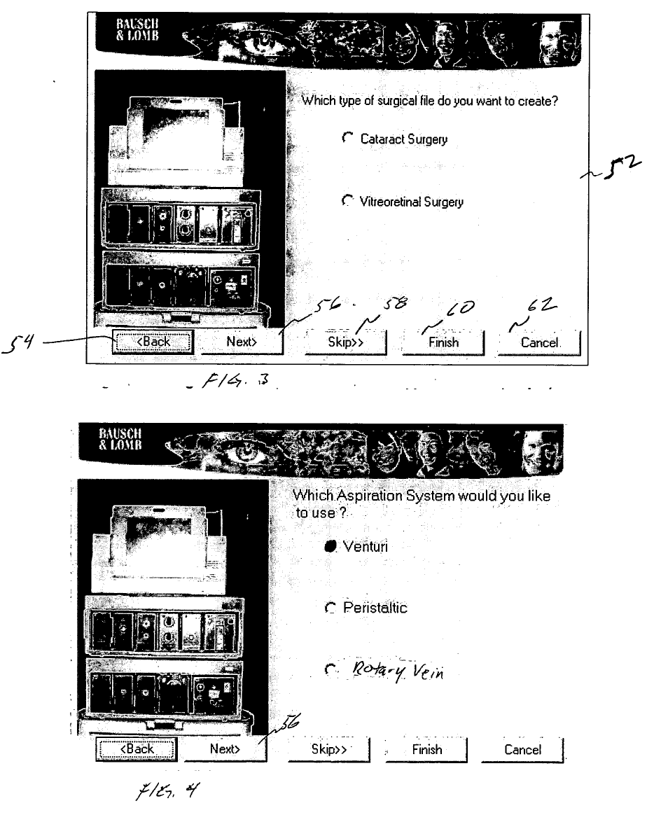

FIG. 2 shows a flow diagram setting up a surgical preference file in accordance with the present invention. Initially, a user will be presented with a screen that's shown in FIG. 3 on display 44. The display of FIG. 3 asks which type of surgical file the user wants to create and gives the user the option of cataract surgery or vitreoretinal surgery.

The user selects in this example “cataract surgery” at screen 52. This selection may be made through the use of a touchscreen or a cursor control device, such as a mouse or a keyboard of system 10 (not shown), or any other known means including physical buttons that may be associated with a particular portion of a screen, as is known.

It is also noted that the screen of FIG. 3. has a set of buttons that are standard and preferably available to the user at all times. These buttons include a back button 54 that sends the system control back the previous screen, allowing modification to previous inputs. Next button 56 sends control to the next input screen in the current prompt sequence. Skip button 58, skips the remaining prompts for the current topic and control continues at the next topic with any unanswered inputs set to default settings. Finish button 60 completes the creation of the user file and uses the default settings for all unanswered inputs. Finally, cancel button 62 aborts creation of the file and disregards inputs and returns control to the initial screen, and in this particular case, we are at initial screen 52. Buttons 54-62 may be soft buttons as shown or could be hard buttons as is known in the prior art, depending on the desired user input mechanism of the system.

After initial screen 52, where the user selects “cataract surgery”, the flow chart of FIG. 2 prompts the user to select an aspiration type at step 64 and as shown in FIG. 4. The user then selects “venturi” and presses the next button 56. Next the system prompts the user by asking whether an anterior file is to be created, shown at step 66. System 10 through user interface 48 then prompts the user at step 68 by asking if the user wishes to customize modes. The user then selects “yes” and presses next button via a screen not shown, but similar to FIGS. 3 and 4. Next at step 70, user interface 48 asks the user if the I/A (irrigation and aspiration) settings are to be customized. The user responds “yes” and presses next button 56. System 10 then queries the user whether custom aspiration settings for I/A mode 1 are to be made; the user selects “yes” and presses next button 56.

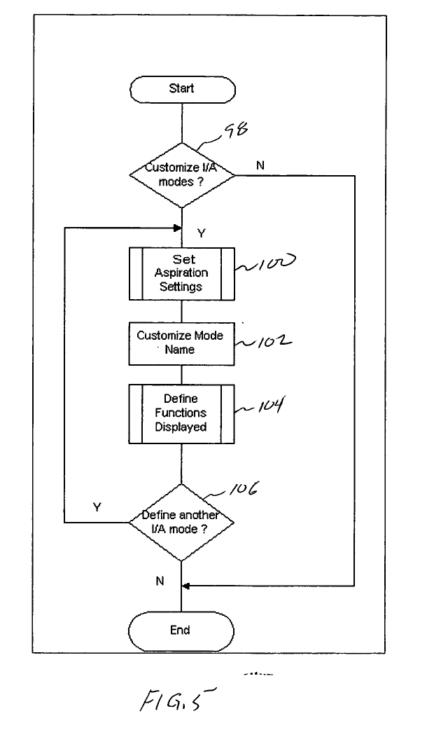

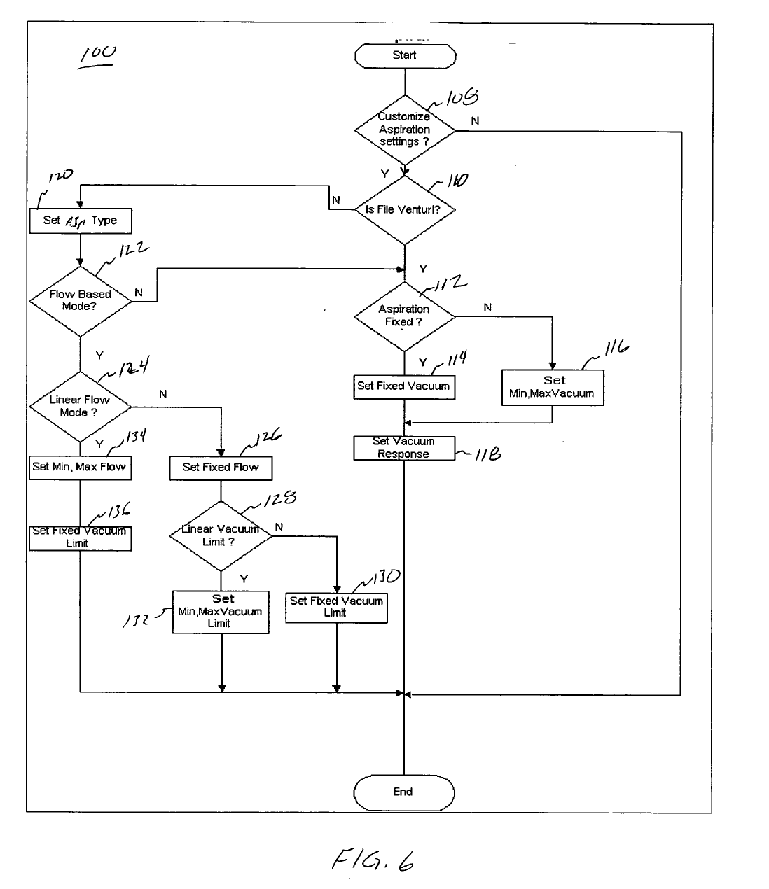

Detailed flow charts of customizing the I/A settings are shown below at FIGS. 5 and 6. Next, the system 10 prompts the user to select fixed aspiration or linear aspiration, and the user selects “linear” and then presses next button 56. The next screen asks the user to enter the aspiration settings, including a minimum aspiration and a maximum aspiration preferably in millimeters of mercury (mmHg) and the user selects “0” for the minimum aspiration and “550” for the maximum aspiration and presses the next button 56.

The system 10 then asks the user to select a vacuum response time, typically presented to the user on a scale such as 1-5 with 1 representing the fastest response and 5 the slowest response. In this example, the user selects “3” and presses next. Obviously, the system depending on the design criteria could present this question in other manners and still be within the scope of the present invention. The system then asks the user to enter a name for this particular I/A mode 1 he has just established; the user inserts a name of his choice via any number of data input means, such as a keyboard or a soft keyboard presented on the screen or through voice recognition and selects next. The system then asks the user if more I/A modes are to be customized, and the user selects “no” and presses next.

The user is then prompted and asked if the I/A pole is to be displayed in I/A mode 1, and the user selects “yes” and presses next. The system then asks the user to entered the bottle height for the I/A, and the user enters “80 centimeters” and presses next. Upon completion of the setup presentation this will cause motor 30 to move pole 28 so that bottle 20 is 80 centimeters above the operating table.

The system 10 then asks if fixed coagulation should be displayed for I/A mode 1, the user selects “yes” and presses next. The system then prompts the user to enter the fixed coagulation setting, and the user enters “12%” and selects skip button 58. As described above, selecting skip 58 will skip the rest of the current function for the coagulation settings, prompting the system to input default settings for the rest of the coagulation information needed.

The system 10 then asks the user if another I/A mode is to be customized, and the user selects “no” and presses next.

User interface 48 then asks if ultrasound (U/S) modes are to be customized, and the user selects “yes” and presses next. The user is then asked if the aspiration settings in U/S mode 1 are to be customized, and the user selects “yes” and presses next. The user is then asked if the aspiration type is linear or fixed, and the user selects “linear” and presses next. The user is then asked to input the aspiration settings, and the user inputs “0” as the minimal aspiration and “400” as the maximum aspiration and selects next. The user is then asked if burst mode is to be enabled, and the user selects “no” and presses next.

The system then asks the user what ultrasound type is to be used, linear or fixed. The user selects “linear ultrasound” and presses next. The system then asks the user to input the ultrasound settings, and the user enters “0%” as a minimal ultrasound setting and “20%” as the maximum ultrasound setting and presses next. The user is then asked to input a desired pulse rate in pulses per second, and the user enters “0” and selects next.

The user is then prompted and asked if the user would like to customize the functions displayed for U/S mode 1. The user selects “no” and presses next. This causes the default settings to be used as to the displayed functions in U/S mode 1.

The user is then prompted to insert a rise time for U/S mode 1 and the user selects “1” and presses next.

The user is then asked if the function of a rocker switch on foot pedal 32 is to be customized for U/S mode 1. The user selects “yes” and presses next. The screen then asks the user to select a desired function for the rocker switch. The options available to the user could be such as the following; U/S power, maximum vacuum, I/V bottle height, modes sequence, or no function. In this particular example, the user selects “maximum vacuum” and presses next.

The user is then requested to insert a mode name and the user inserts “U/S mode 1” or other name of the user's choice.

The user is then asked if another U/S mode is to be customized and the user selects “no” and presses next.

The user is then asked if the vit modes (vitrectomy modes) are to be customized, the user selects “no” and presses next.

The system then prompts the user by asking if foot control settings are to be customized. The user selects “yes” and presses next. The system then asks the user if the foot control detent settings are to be customized. The user selects “yes” and presses next.

The system then requests the user to input the detent settings and the user enters “5, 30, and 50” for positions of the foot pedal and presses next. The system 10 then asks whether the left foot or right foot will be used and the user selects “right foot” and presses next.

The system then asks whether the reflux settings are to be customized and the user selects “skip”. This causes the remaining foot control options to be skipped and default settings used where required.

The system then asks if the start-up mode is to be customized and the user selects “no” and presses next. System 10 then asks if the audio settings are to be customized and the user selects “no” and presses next. The system then asks if the language settings are to be customized and the user selects “no” and presses next.

The system then asks the user to enter a file name and the user enters the desired name and then presses finish button 60. Thus, has been shown an example of how the present invention guides a user through setting up a surgical file and also filters out options at various points.

It is also desired that an expert mode be available to the user so that, for an experienced user, the need for following along the system guided questions is not necessary. In this mode, all options would be available to the user, and the user could then set the system directly as he desires without the aid of the user interface prompting the user with a series of questions.

While the set-up of a surgical file has been shown, other functions and files may be set-up using a similar user interface, such as allowing the user to modify current surgical settings or to set-up user preference files for use in surgical mode. In addition, support functions such as allowing the user to obtain information pertaining to the configuration settings or with regard to setting up a help presentation for the user, file operations for back-up or restoration support and system upgrade procedures may also use a similar user interface guide as described above.

Referring back to FIG. 2, step 72 allows the customization of ultrasound (U/S) settings to be made, such as described above. Step 74 allows customization of vitrectomy settings to be made. If at step 66, the file is not an anterior file, step 76 then asks if customized mode settings are to be made; if yes, step 78 allows customized U/S settings to be made. Step 80 customizes vitrectomy settings, step 82 customizes scissor settings, step 84 customizes coagulation settings, and step 86 customizes viscous fluid settings. If the answer to steps 68 or 76 is “no” or after steps 74 or 86, common customization steps are entered at steps 88-96.

Step 88 allows foot control settings to be customized, step 90 customizes mode sequence settings, step 92 customizes audio settings, step 94 customizes language settings, and step 96 allows the file name to be customized.

FIGS. 5, 6, and 7 set forth flow diagrams for I/A settings and U/S settings.

When a screen presents the user with I/A setting prompts, a logic flow such as shown in FIG. 5 can be used, where at step 98 the system asks the user if the I/A settings are to be customized. If “yes”, step 100 described in detail below at FIG. 6 sets the aspiration setting, step 102 allows the user to name the customized mode, step 104 then allows the user to define the functions to be displayed on display 44, and step 106 asks the user if another I/A mode should be defined. If “yes”, the logic loops back to set step 100. If “no”, I/A customization ends.

FIG. 6 is a detailed logic flow of step 100 and shows a method of customizes aspiration settings, where at step 108 the user is asked if aspiration settings are to be customized. If “yes”, step 110 determines if the aspiration system is for a venturi pump. If “yes”, step 112 asks the user if the aspiration is to be fixed. If step 112 is “yes”, step 114 requests the user to set the fixed vacuum level. If the answer to 112 is “no” the user is requested at step 116 to set the minimum and maximum vacuum levels. Step 118 then asks the user to set the vacuum response as described above.

If the answer to step 110 is “no”, step 120 asks the user to set the aspiration type. Based on the setting of step 120, step 122 determines if the aspiration type is flow based. If “no”, the logic flow proceeds to step 112 as described above. If the answer to step 122 is “yes”, step 124 determines if the user has selected linear flow mode. If “no”, step 126 requests the user to set the fixed flow rate. Step 128 then determines if there is a linear vacuum limit. If “no”, step 130 requests the user to set a fixed vacuum limit. If the answer to step 128 is “yes”, step 132 requests the user to set a minimum/maximum vacuum limit.

If the answer to step 124 is “yes”, step 134 requests the user to set minimum and maximum flow rates. Next, step 136 requests the user to set the fixed vacuum limit. After steps 118, 130, 132, or 136 logic flow ends and proceeds back to step 102 of FIG. 5.

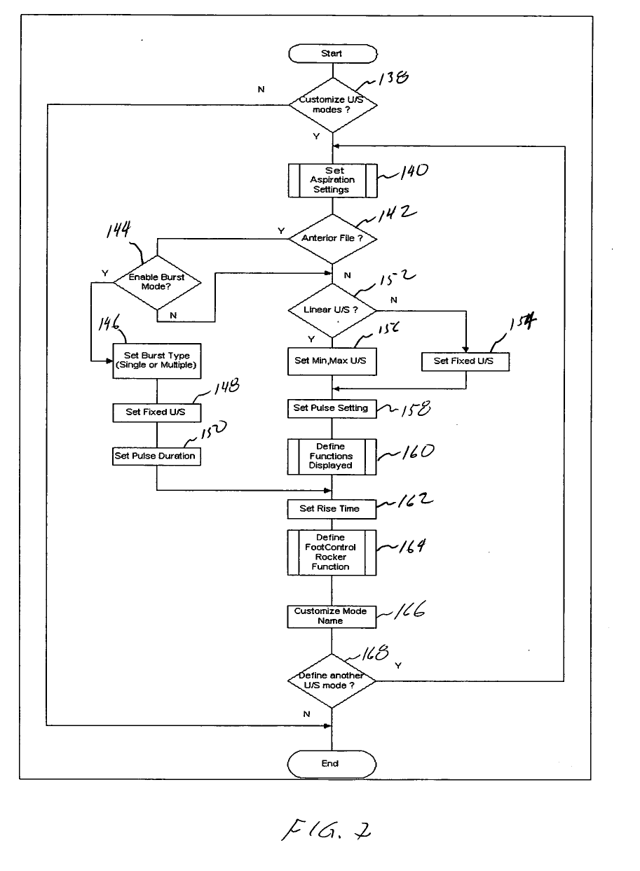

FIG. 7 is a logic flow diagram for customizing U/S settings such as required at steps 72 or 78 of FIG. 2.

If at step 138 the user chooses to customize ultrasound settings, step 140 then sets the aspiration settings, such as described above with respect to FIG. 6. Step 142 then determines if the file being set-up is an anterior file. If “yes”, step 144 asks the user if burst mode is to be used. If “yes”, step 146 asks the user to set the burst mode type, typically single or multiple bursts. Step 148 then asks the user to set the fixed ultrasound setting and step 150 asks the user to set the pulse duration, typically in milliseconds. If the answer to step 142 or step 144 is “no”, step 152 asks the user if linear ultrasound is to be used. If “no”, step 154 asks the user to set the fixed ultrasound setting. If step 152 is “yes”, step 156 asks the user to set the minimum and maximum ultrasound levels. After steps 154 and 156, step 158 asks the user to set the pulse settings. Step 160 then allows the user to define the functions to be displayed. Step 162 asks the user to set the rise time for the ultrasound device. Step 164 allows the foot control rocker switch to be defined, as described above. Step 166 allows a customized mode name to be entered by the user. Finally, step 168 asks the user whether another ultrasound mode is to be defined.

Thus, has been shown detailed examples of how to customize aspiration and ultrasonic settings. Other settings may be defined and the system will guide the user through other settings in a similar fashion but not set forth in detail here. These other settings through which the inventive user face herein may guide a user through, includes customizing vitrectomy settings, coagulation settings, scissor settings, foot control settings, mode sequence settings, audio settings, language settings, rocker switch settings, or customized functions displayed settings. Thus, has been shown and described an inventive guide and filtering presentation to a user for controlling the functions or an ophthalmic surgical system.

1. An ophthalmic surgical system comprising:
at least one aspiration device for aspirating fluids and tissue from a surgical site;
at least one surgical handpiece for performing a surgical function;
a control unit connected to each of the aspiration device and surgical handpiece for controlling the operation of each device and handpiece; and
a user interface forming a portion of the control unit for guiding a user by prompting the user to answer a series of questions and the control unit controlling the devices and handpieces based on the user's answers.

2. The invention of claim 1, wherein the user interface function includes a filtering presentation of options to the user based on the answers provided by the user.

3. The invention of claim 1, wherein the aspiration device includes a peristaltic pump.

4. The invention of claim 1, wherein the aspiration device includes a venturi pump.

5. The invention of claim 1, wherein the aspiration devices includes a rotary vein pump.

6. The invention of claim 1, wherein the surgical handpiece includes one or more taken from a group consisting of an irrigation, ultrasonic, aspiration, coagulator, laser, vitrectomy cutter, scissors, and illumination handpiece.

7. The invention of claim 1, wherein the control unit includes a central processing unit and memory.

8. The invention of claim 1 further including a foot controller having settings and functions defined by the control unit based on answers provided by the user.

9. The invention of claim 1, wherein the user interface further includes an expert mode that disables the prompting of questions and allows the user to select options and make settings directly.

10. A method of controlling an ophthalmic surgical system comprising the steps of:
providing at least one aspiration device for aspirating fluids and tissue from a surgical site;
providing at least one surgical handpiece for performing a surgical function;
connecting a control unit to each aspiration device and surgical handpiece to control the operation of each device and handpiece; and
prompting a user via a user interface contained within a portion of the control unit with a series of questions and controlling the devices and handpieces based on the user's answers.

11. The method of claim 10, wherein the prompting step further includes a filtering presentation of options to the user based on the answers provided by the user.

12. The invention of claim 10, wherein the aspiration device includes a peristaltic pump.

13. The invention of claim 10, wherein the aspiration devices includes a rotary vein pump.

14. The invention of claim 10, wherein the surgical handpiece includes one or more taken from a group consisting of an irrigation, ultrasonic, aspiration, coagulator, laser, vitrectomy cutter, scissors, and illumination handpiece.

15. The invention of claim 10, wherein the control unit includes a central processing unit and memory.

16. The invention of claim 10 further including a foot controller having settings and functions defined by the control unit based on answers provided by the user.

17. The invention of claim 10, wherein the user interface further includes an expert mode that disables the prompting of questions and allows the user to select options and make settings directly.

18. An ophthalmic surgical system comprising:
a plurality of surgical devices and handpieces for performing various surgical functions;
a control unit connected to each device and handpiece for controlling the operation of each device and handpiece, the control unit including memory;
a user interface forming a portion of the control unit for prompting a user to answer a series of questions for guiding the user through a particular procedure available in the system.

19. The system of claim 18, wherein the procedure includes guiding the user to modify surgical settings files.

20. The system of claim 18, wherein the procedure includes guiding the user to select user preference files for use in surgery.

21. The system of claim 18, wherein the procedure includes guiding the user to obtain system information regarding surgical settings, file operations, and system upgrade procedures.

22. The system of claim 18, wherein the procedure includes guiding the user during surgery to provide the user with help information regarding the use of the system.

23. The invention of claim 18, wherein the user interface function includes a filtering presentation of options to the user based on the answers provided by the user.

24. The invention of claim 18, wherein the aspiration device includes a peristaltic pump.

25. The invention of claim 18, wherein the aspiration device includes a venturi pump.

26. The invention of claim 18, wherein the aspiration devices includes a rotary vein pump.

27. The invention of claim 18, wherein the surgical handpiece includes one or more taken from a group consisting of an irrigation, ultrasonic, aspiration, coagulator, laser, vitrectomy cutter, scissors, and illumination handpiece.

28. The invention of claim 18, wherein the control unit includes a central processing unit and memory.

29. The invention of claim 18 further including a foot controller having settings and functions defined by the control unit based on answers provided by the user.

30. The invention of claim 18, wherein the user interface further includes an expert mode that disables the prompting of questions and allows the user to select options and make settings directly.
